(19) United States
(12) Patent Application Publication
(10) Pub. No.: US 2026/0182922 A1
(43) Pub. Date: Jul. 2, 2026

(54) METHOD AND DEVICE FOR GENERATING TRAINING DATASET OF BIO-SIGNAL DENOISING AI MODEL

(71) Applicant: Electronics and Telecommunications Research Institute, Daejeon (KR)

(72) Inventors: Youngwoong HAN, Daejeon (KR); Hyung Wook NOH, Sejong-si (KR); Seohee SO, Daejeon (KR); Myung-eun LIM, Daejeon (KR); Ho-Youl JUNG, Daejeon (KR)

(73) Assignee: Electronics and Telecommunications Research Institute, Daejeon (KR)

(21) Appl. No.: 19/322,782

(22) Filed: Sep. 9, 2025

(30) Foreign Application Priority Data
Dec. 26, 2024 (KR) 10-2024-0197589

(51) Int. Cl. A61B 5/00; G06F 18/2134

(52) U.S. Cl. CPC A61B 5/7203; A61B 5/7267; G06F 18/2134

(57) ABSTRACT

A method and device for generating a training dataset of a bio-signal denoising artificial intelligence (AI) model are provided. According to an embodiment, the method includes receiving first bio-signal data measured in a non-noise-suppressed environment and second bio-signal data measured in a noise-suppressed environment, performing component analysis of the first bio-signal data, and determining noise component data by separating target component data from the first bio-signal data, generating third bio-signal data by combining the noise component data with the second bio-signal data, and determining the second bio-signal data as ground truth data of the training dataset and determining the third bio-signal data as noisy data of the training dataset to determine the training dataset.

### CROSS-REFERENCE TO RELATED APPLICATION

This application claims the benefit of Korean Patent Application No. 10-2024-0197589 filed on Dec. 26, 2024, in the Korean Intellectual Property Office, the entire disclosure of which is incorporated herein by reference for all purposes.

### BACKGROUND

### 1. Field

The disclosure relates to a method and a device for generating a training dataset of a bio-signal denoising artificial intelligence (AI) model.

### 2. Description of the Related Art

Bio-signals are electrical, chemical, or physical signals generated by the body. Bio-signals may include, for example, electroencephalogram (EEG), electrooculogram (EOG), electrocardiogram (ECG), electromyogram (EMG), and the like. When a bio-signal is measured in a living organism, bio-signals other than the desired bio-signal may be measured as noise. Such noise may be measured even during relax time, when a user is inactive.

### SUMMARY

It may be difficult to accurately measure or extract other bio-signals from bio-signal data measured using a bio-signal measurement device. When ground truth data in a training dataset of a bio-signal denoising artificial intelligence (AI) model includes a lot of noise, or data in the training dataset including noise does not include the ground truth data as is, the performance of the bio-signal denoising AI model trained with the training dataset may be degraded.

According to an aspect of the disclosure, a method of generating a training dataset of a bio-signal denoising AI model includes receiving first bio-signal data measured in a non-noise-suppressed environment and second bio-signal data measured in a noise-suppressed environment, performing component analysis of the first bio-signal data, and determining noise component data by separating target component data from the first bio-signal data, generating third bio-signal data by combining the noise component data with the second bio-signal data, and determining the second bio-signal data as ground truth data of the training dataset and determining the third bio-signal data as noisy data of the training dataset to determine the training dataset.

According to an aspect of the disclosure, a training method of a bio-signal denoising AI model includes receiving first bio-signal data measured in a non-noise-suppressed environment and second bio-signal data measured in a noise-suppressed environment, performing component analysis of the first bio-signal data, and determining noise component data by separating target component data from the first bio-signal data, generating third bio-signal data by combining the noise component data with the second bio-signal data, determining the second bio-signal data as ground truth data of a training dataset of the denoising AI model and determining the third bio-signal data as noisy data of the training dataset to determine the training dataset, generating fourth bio-signal data by executing the denoising AI model based on the noisy data and removing noise data included in the noisy data, determining a loss value based on a difference between the ground truth data and the fourth bio-signal data or based on a difference between the noise component data and the noise data, and training the denoising AI model based on the loss value.

According to an aspect of the disclosure, a device for generating a training dataset of a bio-signal denoising AI model includes one or more processors, and memory including instructions executable by the one or more processors, wherein the instructions, when executed by the one or more processors, may cause the device to receive first bio-signal data measured in a non-noise-suppressed environment and second bio-signal data measured in a noise-suppressed environment, perform component analysis of the first bio-signal data, and determine noise component data by separating target component data from the first bio-signal data, generate third bio-signal data by combining the noise component data with the second bio-signal data, and determine the second bio-signal data as ground truth data of the training dataset and determine the third bio-signal data as noisy data of the training dataset to determine the training dataset.

Additional aspects of one or more embodiments will be set forth in part in the description which follows and, in part, will be apparent from the description, or may be learned by practice of the disclosure.

According to one or more embodiments, a training dataset capable of training a denoising AI model without degrading performance of the denoising AI model may be generated.

### BRIEF DESCRIPTION OF THE DRAWINGS

The above and other aspects and features of certain embodiments of the present disclosure will be more apparent from the following description taken in conjunction with the accompanying drawings, in which:

FIG. 1A is a diagram illustrating an example training process of a typical bio-signal denoising artificial intelligence (AI) model, according to an embodiment;

FIG. 1B is a diagram illustrating an example training process of a bio-signal denoising AI model that is a conditional diffusion model, according to an embodiment;

FIG. 2 is a block diagram schematically illustrating an operation of a training dataset generation device, according to an embodiment;

FIG. 3 is a flowchart illustrating a method of generating a training dataset of a bio-signal denoising AI model based on noise-suppressed bio-signal data and non-noise-suppressed bio-signal data, according to an embodiment;

FIG. 4 is a diagram illustrating an example process of generating noisy data of a training dataset based on noise-suppressed bio-signal data and non-noise-suppressed bio-signal data, according to an embodiment;

FIG. 5 is a flowchart illustrating a method of generating a training dataset of a denoising AI model and training the denoising AI model using the training dataset, according to an embodiment;

FIG. 6 is a flowchart illustrating a method of generating a training dataset of a bio-signal denoising AI model, according to an embodiment; and

FIG. 7 is a block diagram illustrating a configuration of an electronic device generating a training dataset of a bio-signal denoising AI model, according to an embodiment.

### DETAILED DESCRIPTION

The following detailed structural or functional description is provided as an example only and various alterations and modifications may be made to the embodiments described herein. Accordingly, the embodiments described herein are not intended to limit the disclosure and should be understood to include all changes, equivalents, and replacements within the idea and the technical scope of the disclosure.

Although terms of “first,” “second,” and the like are used to explain various components, the components are not limited to the terms. These terms should be used only to distinguish one component from another component. For example, a first component may be referred to as a second component, and similarly, the second component may also be referred to as the first component.

It will be understood that when a component is referred to as being “connected to” or “coupled” to another component, the component may be directly connected or coupled to the other component or intervening components may be present.

As used herein, the singular forms “a,” “an,” and “the” are intended to include the plural forms as well, unless the context clearly indicates otherwise. It will be further understood that the terms “comprises/comprising” and/or “includes/including” when used herein, specify the presence of stated features, integers, steps, operations, elements, and/or components, but do not preclude the presence or addition of one or more other features, integers, steps, operations, elements, components and/or groups thereof.

Unless otherwise defined, all terms, including technical and scientific terms, used herein have the same meaning as commonly understood by one of ordinary skill in the art to which this disclosure pertains. Terms, such as those defined in commonly used dictionaries, are to be interpreted as having a meaning that is consistent with their meaning in the context of the relevant art, and are not to be interpreted in an idealized or overly formal sense unless expressly so defined herein.

Hereinafter, embodiments will be described in detail with reference to the accompanying drawings. When describing the embodiments with reference to the accompanying drawings, like reference numerals refer to like components and a repeated description related thereto will be omitted.

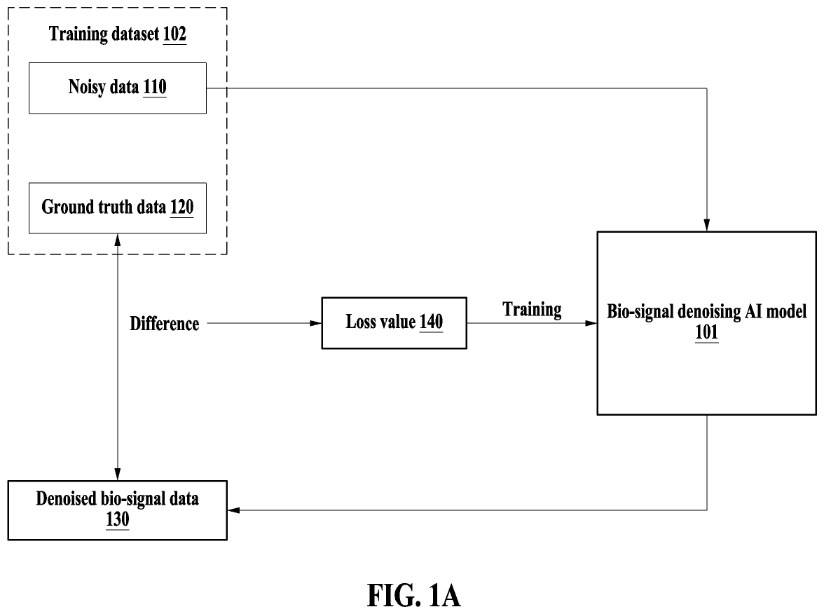

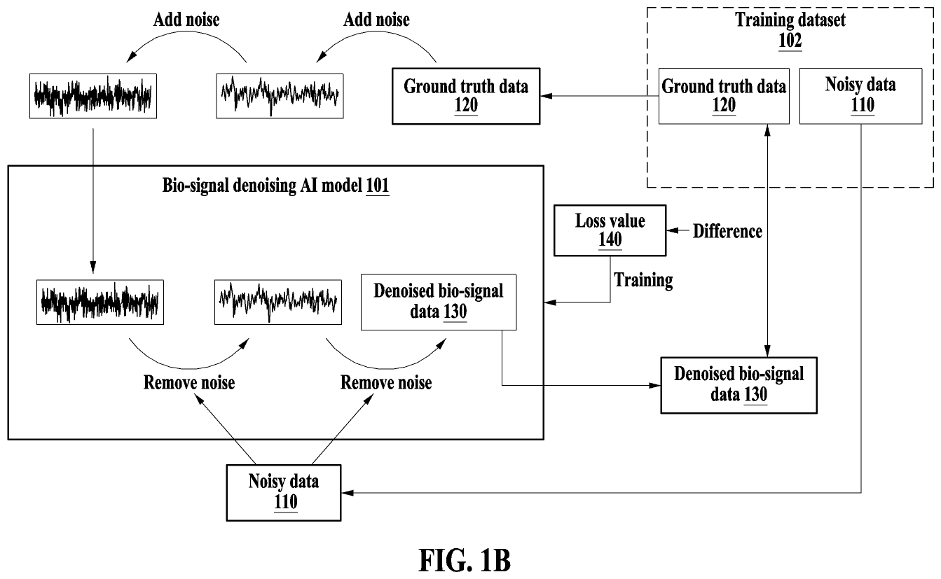

Bio-signals (e.g., electroencephalograms (EEGs)) may be measured by a bio-signal measurement device (e.g., an EEG measurement device, a wearable measurement device and a body-non-contact measurement device such as LiDAR). Typically, bio-signal data measured by a bio-signal measurement device may be contaminated with noise. Noise that contaminates bio-signals may also be referred to as an artifact. Since noise may be a factor in that reduces the reliability of bio-signal analysis, bio-signal analysis may be performed based on data from which noise has been removed using a denoising artificial intelligence (AI) model. A bio-signal denoising AI model may be an AI model for denoising bio-signal data measured by a bio-signal measurement device. The bio-signal denoising AI model may remove noise including bio-signals (e.g., when a target bio-signal is an EEG, for example, an electrooculogram (EOG)) other than the target bio-signal (e.g., an EEG), and/or external noise (e.g., noise from automobiles). The following description of FIGS. 1A and 1B may be examples of a training process of the bio-signal denoising AI model.

FIG. 1A is a diagram illustrating an example training process of a typical bio-signal denoising AI model, according to an embodiment. Referring to FIG. 1A, a bio-signal denoising AI model 101 may be trained through supervised learning using a training dataset 102. The training dataset 102 may include noisy data 110 and ground truth data 120. The noisy data 110 may correspond to bio-signal data contaminated with noise. The ground truth data 120 may correspond to bio-signal data from which noise has been removed.

The bio-signal denoising AI model 101 may receive the noisy data 110 as an input. The bio-signal denoising AI model 101 may remove noise from the noisy data 110. The bio-signal denoising AI model 101 may generate denoised bio-signal data 130 based on the noisy data 110. To train the bio-signal denoising AI model 101, a loss value 140 may be determined based on a difference between the ground truth data 120 and the denoised bio-signal data 130. The bio-signal denoising AI model 101 may be trained based on the loss value 140.

FIG. 1B is a diagram illustrating an example training process of a bio-signal denoising AI model that is a conditional diffusion model, according to an embodiment. Referring to FIG. 1B, the bio-signal denoising AI model 101 may be trained through a forward process and a reverse process using the training dataset 102. The bio-signal denoising AI model 101 of FIG. 1B may correspond to a conditional diffusion model.

To train the bio-signal denoising AI model 101, noise may be gradually added to the ground truth data 120. The noise gradually added may be gaussian noise. The process of gradually adding noise to the ground truth data 120 may correspond to a forward process. Data to which noise is added through the forward process may be input to the bio-signal denoising AI model 101.

The bio-signal denoising AI model 101 may gradually remove noise from data input to the bio-signal denoising AI model 101. The bio-signal denoising AI model 101 may use the noisy data 110 as a condition when gradually removing noise from the input data. The bio-signal denoising AI model 101 may gradually remove noise from the input data to generate the denoised bio-signal data 130.

The loss value 140 may be determined to train the bio-signal denoising AI model 101. The loss value 140 may be determined based on a difference between the ground truth data 120 and the denoised bio-signal data 130. In addition, although not shown in FIG. 1B, a loss value may be determined based on a difference between estimated noise data and noise data included in the noisy data 110. The estimated noise data may correspond to data estimated as noise and removed by the bio-signal denoising AI model 101 when the bio-signal denoising AI model 101 gradually removes noise from input data. The noise data included in the noisy data 110 may correspond to a difference between the noisy data 110 and the ground truth data 120. The bio-signal denoising AI model 101 may be trained based on the loss value 140.

As described above with reference to FIGS. 1A and 1B, training a bio-signal denoising AI model may require a training dataset including noisy data and ground truth data. The quality of a training dataset may directly impact the performance of a bio-signal denoising AI model.

Considering the characteristics of bio-signals, to ensure the denoising performance of a bio-signal denoising AI model, ground truth data in the training dataset may have to be data in which noise is maximally suppressed, excluding the target bio-signal. Additionally, to ensure the denoising performance of the bio-signal denoising AI model, the noisy data in the training dataset may have to be data that includes noise in the ground truth data, rather than random data. Due to the characteristics of bio-signal data, it may be difficult to acquire ground truth data in which noise is maximally suppressed and noisy data that includes the ground truth data as is.

The disclosure may provide a method and device for generating a training dataset of a bio-signal denoising AI model using data measured by a single bio-signal measurement device without using external data.

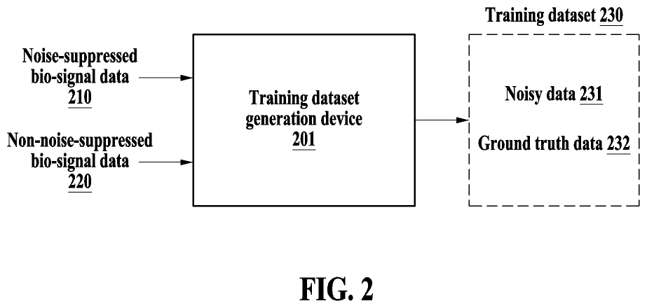

FIG. 2 is a block diagram schematically illustrating an operation of a training dataset generation device, according to an embodiment. Referring to FIG. 2, a training dataset generation device 201 may receive noise-suppressed bio-signal data 210 and non-noise-suppressed bio-signal data 220 as an input. The noise-suppressed bio-signal data 210 may be bio-signal data measured in a noise-suppressed environment. The non-noise-suppressed bio-signal data 220 may be bio-signal data measured in a non-noise-suppressed environment. The noise-suppressed bio-signal data 210 and the non-noise-suppressed bio-signal data 220 may be bio-signal data measured by the same bio-signal measurement device.

In a noise-suppressed environment, noise to be suppressed may include one or more of a bio-signal excluding a target bio-signal to be acquired by executing a denoising AI model among bio-signals measured by a bio-signal measurement device, or external noise. The noise to be suppressed in a noise-suppressed environment may correspond to noise to be removed by the bio-signal denoising AI model through inference after training is completed.

The target bio-signal may be a bio-signal corresponding to, for example, one of an EEG, EOG, electrocardiogram (ECG), and electromyogram (EMG). The external noise may correspond to noise other than a bio-signal. The noise-suppressed environment may be an environment in which one or more of the bio-signal excluding the target bio-signal to be acquired by executing the bio-signal denoising AI model among the measured bio-signals or the external noise is suppressed. For example, when a noise to be removed through the inference of the bio-signal denoising AI model after the training of the bio-signal denoising AI model is completed is external noise generated during operation of an ambulance, a non-noise-suppressed environment may be an ambulance operating environment, and the noise-suppressed environment may be an environment in which an engine is turned off and vibration and noise are suppressed inside the ambulance.

The training dataset generation device 201 may generate a training dataset 230 based on the noise-suppressed bio-signal data 210 and the non-noise-suppressed bio-signal data 220. The training dataset 230 may include noisy data 231 and ground truth data 232. The training dataset 230, noisy data 231, and ground truth data 232 may correspond to the training dataset 102, noisy data 110, and ground truth data 120 of FIGS. 1A and 1B.

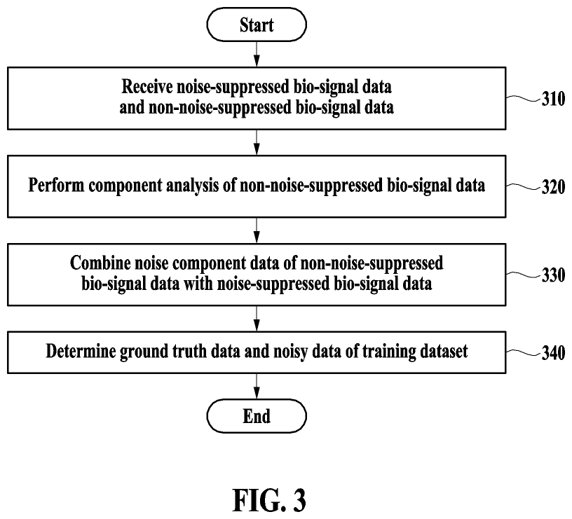

FIG. 3 is a flowchart illustrating a method of generating a training dataset of a bio-signal denoising AI model based on noise-suppressed bio-signal data and non-noise-suppressed bio-signal data, according to an embodiment. Referring to FIG. 3, in operation 310, noise-suppressed bio-signal data and non-noise-suppressed bio-signal data may be received. The noise-suppressed bio-signal data and non-noise-suppressed bio-signal data may correspond to the noise-suppressed bio-signal data 210 and non-noise-suppressed bio-signal data 220 of FIG. 2, respectively.

In operation 320, component analysis may be performed for the non-noise-suppressed bio-signal data. Component analysis may correspond to an interpretation of underlying structural components or latent variables in data. The component analysis may correspond to, for example, independent component analysis (ICA) or principal component analysis (PCA).

By performing component analysis on the non-noise-suppressed bio-signal data, target component data may be separated from the non-noise-suppressed bio-signal data. The target component data may be component data corresponding to a target bio-signal among the component data of the non-noise-suppressed bio-signal data. The data of components other than a target component in the non-noise-suppressed bio-signal data may correspond to noise component data. The noise component data may be determined by performing component analysis on the non-noise-suppressed bio-signal data.

In operation 330, the noise component data of the non-noise-suppressed bio-signal data may be combined with noise-suppressed bio-signal data. Bio-signal data generated by combining the noise component data of the non-noise-suppressed bio-signal data and the noise-suppressed bio-signal data may be bio-signal data including the noise-suppressed bio-signal data as is. The noise component data of the non-noise-suppressed bio-signal data may be selected based on a predetermined selection category before being combined with the noise-suppressed bio-signal data. The combining of the noise component data of the non-noise-suppressed bio-signal data and the noise-suppressed bio-signal data may be performed based on a predetermined signal-to-noise ratio (SNR) value.

In operation 340, ground truth data and noisy data of the training dataset may be determined. The noise-suppressed bio-signal data received in operation 310 may be determined as ground truth data of the training dataset. In operation 330, the bio-signal data generated by combining the noise component data of the non-noise-suppressed bio-signal data and the noise-suppressed bio-signal data may be determined as noisy data of the training dataset. By determining the noise-suppressed bio-signal data as ground truth data and determining the bio-signal data combined in operation 330 as noisy data, the training dataset of the bio-signal denoising AI model may be determined. The noisy data and the ground truth data may correspond to the noisy data 231 and the ground truth data 232 of FIG. 2, respectively.

The ground truth data may be data in which noise is maximally suppressed. The ground truth data may be data in which noise to be removed through the inference of the bio-signal denoising AI model is suppressed. Additionally, since the noisy data is generated by combining the noise component data of the non-noise-suppressed bio-signal data and the ground truth data, the noisy data may be bio-signal data including additional noise component data in the ground truth data. Since generating the training dataset does not require external data and a bio-signal measurement device other than the target bio-signal measurement device, a training dataset for the bio-signal denoising AI model may be generated even in limited situations where external data or a separate bio-signal measurement device is not available.

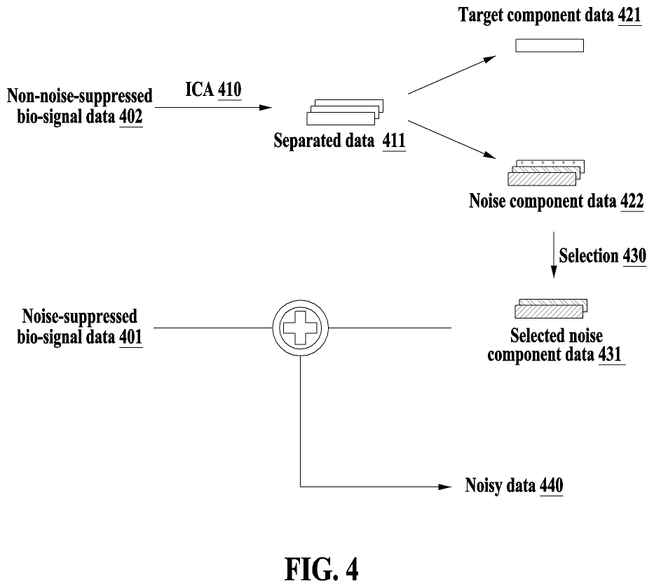

FIG. 4 is a diagram illustrating an example process of generating noisy data of a training dataset based on noise-suppressed bio-signal data and non-noise-suppressed bio-signal data, according to an embodiment. Referring to FIG. 4, an ICA 410 may be performed on non-noise-suppressed bio-signal data 402. The ICA 410 may correspond to the component analysis performed in operation 320 of FIG. 3. The non-noise-suppressed bio-signal data 402 may correspond to the non-noise-suppressed bio-signal data 220 of FIG. 2.

By performing the ICA 410, the non-noise-suppressed bio-signal data 402 may be separated into separated data 411. The ICA 410 may locate and separate components that maximize statistical independence within the data. The ICA 410 may be performed based on the number of predetermined independent component categories. The number of separated data 411 may be equal to the number of predetermined independent component categories.

For example, the predetermined independent component categories may include EEG, EOG, ECG, and EMG. For example, when there are four predetermined independent component categories: EEG, EOG, ECG, and EMG, the non-noise-suppressed bio-signal data 402 may be separated into four pieces of separated data 411.

The separated data 411 may be labeled based on the predetermined independent component categories. For example, the four pieces of separated data 411 may be labeled as EEG data, EOG data, ECG data, and EMG data, respectively. The bio-signal denoising AI model may include a data labeling module. The separated data 411 may be labeled by the data labeling module. The data labeling module may be implemented using an open source data analysis tool. For example, the data labeling module may use an ICLabel tool of EEGLAB, which is an EEG analysis software. The separated data 411 on which labeling is performed may include target component data 421 and noise component data 422. The target component data 421 may be determined from the separated data 411 by labeling. The target component data 421 may be component data corresponding to a target bio-signal among the separated data 411.

The noise component data 422 may be component data excluding the target component data 421 from the separated data 411. The noise component data 422 may be determined by removing the target component data 421 from the non-noise-suppressed bio-signal data 402. Selection 430 may be performed on the noise component data 422. Selected noise component data 431 may be determined based on a predetermined selection category among predetermined independent component categories. For example, when the noise component data 422 is component data corresponding to EOG, ECG, and EMG, the selected noise component data 431 may include component data corresponding to EOG and ECG, but may not include component data corresponding to EMG. When the selection 430 is not performed, the selected noise component data 431 may be the noise component data 422.

Noise-suppressed bio-signal data 401 and the selected noise component data 431 may be combined to generate noisy data 440. The noise-suppressed bio-signal data 401 may correspond to the noise-suppressed bio-signal data 210 of FIG. 2. The noise-suppressed bio-signal data 401 and the selected noise component data 431 may be combined based on a predetermined SNR value. The predetermined SNR value may be a value randomly selected from a plurality of predetermined values. The plurality of predetermined values may correspond to desired SNR values of the noisy data of the training dataset. A weight of the selected noise component data 431 may be determined based on the predetermined SNR value. The noisy data 440 may be generated by adding the selected noise component data 431 multiplied by the weight of the selected noise component data 431 to the noise-suppressed bio-signal data 401.

The weight of the selected noise component data 431 may be determined such that an SNR value of the noisy data 440 calculated based on the noise-suppressed bio-signal data 401 included in the noisy data and the selected noise component data 431 multiplied by the weight is equal to the predetermined SNR value. The SNR value of the noisy data 440 may be a ratio of the noise-suppressed bio-signal data 401 to the noise component data 431 multiplied by the weight. When the SNR value of the noisy data 440 is calculated, a signal may correspond to the noise-suppressed bio-signal data 401 and noise may correspond to the noise component data 431 multiplied by the weight. The SNR value of the noisy data 440 may be determined, for example, through the following Equation 1. SNR of Equation 1 may be equal to the predetermined SNR value. The weight of the noise component data 422 may be determined such that the SNR value of the noisy data 440 is equal to the predetermined SNR value. RMS of Equation 1 may be a calculation formula for the root mean square. k of Equation 1 may be the weight of the noise component data 422. n may correspond to the noise component data 422. t may correspond to the target component data 421.

$$SNR = 10 log \frac{RMS ( t )}{RMS ( k \cdot n )} \qquad [ Equation 1 ]$$

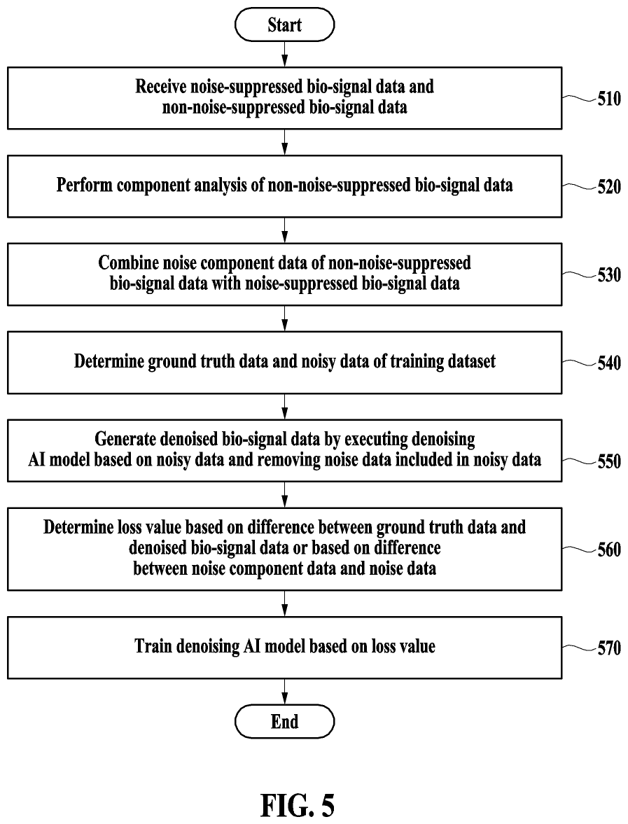

FIG. 5 is a flowchart illustrating a method of generating a training dataset of a denoising AI model and training the denoising AI model using the training dataset, according to an embodiment. Referring to FIG. 5, in operation 510, noise-suppressed bio-signal data and non-noise-suppressed bio-signal data may be received. In operation 520, component analysis of the non-noise-suppressed bio-signal data may be performed. In operation 530, noise component data of the non-noise-suppressed bio-signal data may be combined with the noise-suppressed bio-signal data. In operation 540, ground truth data and noisy data of a training dataset may be determined. Operations 510 to 540 may correspond to operations 310 to 340 of FIG. 3.

In operation 550, a denoising AI model may be executed based on the noisy data to remove noise data included in the noisy data and generate denoised bio-signal data. The denoising AI model may be a bio-signal denoising AI model. The denoising AI model may receive noisy data as an input. The denoising AI model may perform inference to estimate and remove noise from the noisy data, and thereby generate denoised bio-signal data. The noise data included in the noisy data may be data estimated and removed by the denoising AI model. The denoising AI model may use the noisy data as a condition to perform inference to remove noise from bio-signal data to which gaussian noise is added to ground truth data, and thereby generate denoised bio-signal data. The denoising AI model may be an AI model that has not been trained yet. The denoising AI model may be a pre-trained AI model.

In operation 560, a loss value may be determined based on a difference between the ground truth data and the denoised bio-signal data, or based on a difference between the noise component data and noise data. The noise data may be noise data removed by the denoising AI model in operation 550. In operation 570, the denoising AI model may be trained based on the loss value. The denoising AI model may be trained in a direction that reduces the loss value. When the denoising AI model is a pre-trained AI model, the denoising AI model may be fine-tuned using the training dataset.

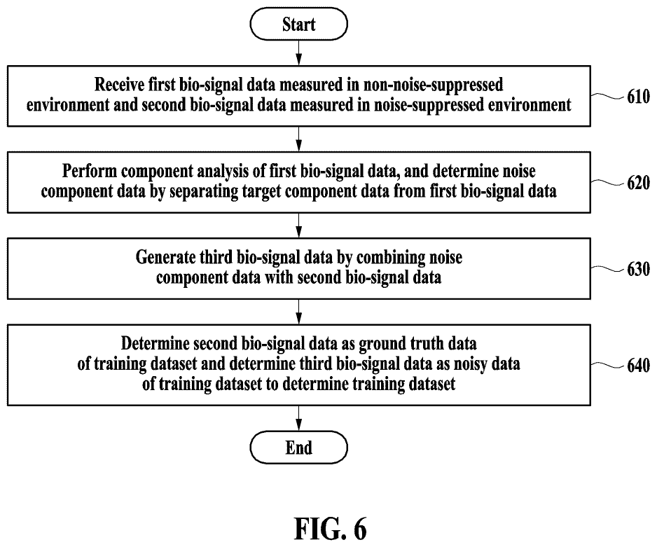

FIG. 6 is a flowchart illustrating a method of generating a training dataset of a bio-signal denoising AI model, according to an embodiment. Referring to FIG. 6, in operation 610, a training dataset generation device may receive first bio-signal data measured in a non-noise-suppressed environment and second bio-signal data measured in a noise-suppressed environment. The noise-suppressed environment may be an environment in which at least one of a bio-signal excluding a target bio-signal to be acquired by executing the denoising AI model among measured bio-signals, or external noise is suppressed. The target bio-signal may correspond to one of an EEG, EOG, ECG, and EMG.

In operation 620, the training dataset generation device may perform component analysis of the first bio-signal data and determine noise component data by separating target component data from the first bio-signal data. The target component data may be component data corresponding to a target bio-signal among component data of the first bio-signal data. The component analysis may correspond to an ICA. The training dataset generation device may separate the first bio-signal data by performing ICA based on the number of predetermined independent component categories. The training dataset generation device may label the separated data based on the predetermined independent component categories. The training dataset generation device may determine noise component data by removing the target component data determined by labeling from the first bio-signal data.

In operation 630, the training dataset generation device may generate third bio-signal data by combining the noise component data with the second bio-signal data. The training dataset generation device may determine a weight of the noise component data based on a predetermined SNR value. The training dataset generation device may generate the third bio-signal data by adding the noise component data multiplied by the weight to the second bio-signal data. The training dataset generation device may determine the weight such that an SNR value of the third bio-signal data is equal to the predetermined SNR value.

In operation 640, the training dataset generation device may determine the second bio-signal data as ground truth data of the training dataset and determine the third bio-signal data as noisy data of the training dataset to determine the training dataset.

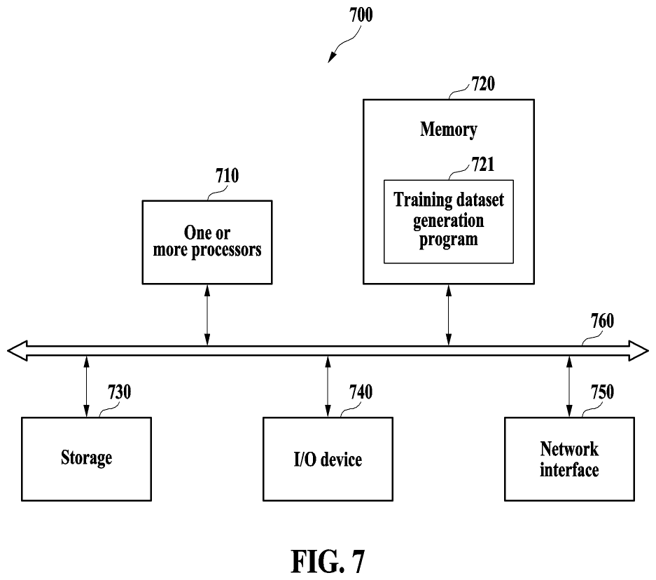

FIG. 7 is a block diagram illustrating a configuration of an electronic device generating a training dataset of a bio-signal denoising AI model, according to an embodiment. Referring to FIG. 7, an electronic device 700 may include one or more processors 710, memory 720, a storage 730, an input/output (I/O) device 740, and a network interface 750, which may communicate with each other via a communication bus 760.

The one or more processors 710 may execute instructions stored in the memory 720 or storage 730. The instructions, when executed by the one or more processors 710, may cause the electronic device 700 to perform the operations described with reference to FIGS. 1A to 6. The memory 720 may include a non-transitory computer-readable storage medium or non-transitory a computer-readable storage device. The memory 720 may store instructions to be executed by the one or more processors 710 and may store related information while software and/or applications are executed by the electronic device 700. The memory 720 may store a training dataset generation program 721 that generates a training dataset of a bio-signal denoising AI model according to an embodiment. In a state where at least a portion of the training dataset generation program 721 is stored in the memory 720, the electronic device 700 may perform the operations described with reference to FIGS. 1A to 6.

The storage 730 may include a non-transitory computer-readable storage medium or a non-transitory computer-readable storage device. The storage 730 may store a greater amount of information than the memory 720 and may store the information for a longer period of time. For example, the storage 730 may include a magnetic hard disk, an optical disk, flash memory, a floppy disk, or any other form of non-volatile memory known in the art.

The I/O device 740 may receive an input from a user via traditional input schemes such as a keyboard and mouse, and via new input schemes such as a touch input, voice input, and image input. For example, the I/O device 740 may include a keyboard, a mouse, a touch screen, a microphone, or any other device configured to detect an input from a user and transmit the detected input to the electronic device 700. The I/O device 740 may provide an output of the electronic device 700 to the user via visual, auditory, or tactile channels. The I/O device 740 may include, for example, a display, a touch screen, a speaker, a vibration generation device, or any other device capable of providing an output to the user. The network interface 750 may communicate with an external device via a wired or wireless network.

The components described in the embodiments may be implemented by hardware components including, for example, at least one digital signal processor (DSP), a processor, a controller, an application-specific integrated circuit (ASIC), a programmable logic element, such as a field programmable gate array (FPGA), other electronic devices, or combinations thereof. At least some of the functions or the processes described in the embodiments may be implemented by software, and the software may be recorded on a recording medium. The components, the functions, and the processes described in the embodiments may be implemented by a combination of hardware and software.

The embodiments described herein may be implemented using a hardware component, a software component and/or a combination thereof. A processing device may be implemented using one or more general-purpose or special-purpose computers, such as, for example, a processor, a controller and an arithmetic logic unit (ALU), a DSP, a microcomputer, an FPGA, a programmable logic unit (PLU), a microprocessor or any other device capable of responding to and executing instructions in a defined manner. The processing device may run an operating system (OS) and one or more software applications that run on the OS. The processing device also may access, store, manipulate, process, and generate data in response to execution of the software. For purpose of simplicity, the description of a processing device is singular; however, one of ordinary skill in the art will appreciate that a processing device may include a plurality of processing elements and a plurality of types of processing elements. For example, the processing device may include a plurality of processors, or a single processor and a single controller. In addition, different processing configurations are possible, such as parallel processors.

The software may include a computer program, a piece of code, an instruction, or some combination thereof, to independently or uniformly instruct or configure the processing device to operate as desired. Software and/or data may be embodied permanently or temporarily in any type of machine, component, physical or virtual equipment, or computer storage medium or device capable of providing instructions or data to or being interpreted by the processing device. The software also may be distributed over network-coupled computer systems so that the software is stored and executed in a distributed fashion. The software and data may be stored by one or more non-transitory computer-readable recording mediums.

The methods according to the above-described embodiments may be recorded in non-transitory computer-readable media including program instructions to implement various operations of the above-described embodiments. The media may also include, alone or in combination with the program instructions, data files, data structures, and the like. The program instructions recorded on the media may be those specially designed and constructed for the purposes of embodiments, or they may be of the kind well-known and available to those having skill in the computer software arts. Examples of non-transitory computer-readable media include magnetic media such as hard disks, floppy disks, and magnetic tape; optical media such as CD-ROM discs and DVDs; magneto-optical media such as optical discs; and hardware devices that are specially configured to store and perform program instructions, such as read-only memory (ROM), random access memory (RAM), flash memory, and the like. Examples of program instructions include both machine code, such as produced by a compiler, and files containing higher-level code that may be executed by the computer using an interpreter.

The above-described hardware devices may be configured to act as one or more software modules in order to perform the operations of the above-described embodiments, or vice versa.

Although one or more embodiments have been described with reference to the accompanying drawings, one of ordinary skill in the art may apply various technical modifications and variations based thereon. For example, suitable results may be achieved if the described techniques are performed in a different order, and/or if components in a described system, architecture, device, or circuit are combined in a different manner, and/or replaced or supplemented by other components or their equivalents.

Therefore, other implementations, embodiments, and equivalents to the claims are also within the scope of the following claims.

What is claimed is:

1. A method of generating a training dataset of a bio-signal denoising artificial intelligence (AI) model, the method comprising:
receiving first bio-signal data measured in a non-noise-suppressed environment and second bio-signal data measured in a noise-suppressed environment;
performing component analysis of the first bio-signal data, and determining noise component data by separating target component data from the first bio-signal data;
generating third bio-signal data by combining the noise component data with the second bio-signal data; and
determining the second bio-signal data as ground truth data of the training dataset and determining the third bio-signal data as noisy data of the training dataset to determine the training dataset.

2. The method of claim 1, wherein the noise-suppressed environment is
an environment in which at least one of a bio-signal excluding a target bio-signal to be acquired by executing the denoising AI model among measured bio-signals, or external noise is suppressed, and
the target component data is
component data corresponding to the target bio-signal among component data of the first bio-signal data.

3. The method of claim 2, wherein the target bio-signal corresponds to one of an electroencephalogram (EEG), electrooculogram (EOG), electrocardiogram (ECG), and electromyogram (EMG).

4. The method of claim 1, wherein the component analysis corresponds to an independent component analysis (ICA).

5. The method of claim 4, wherein the determining of the noise component data comprises:
separating the first bio-signal data by performing the ICA based on the number of predetermined independent component categories;
labeling the separated data based on the predetermined independent component categories; and
determining the noise component data by removing the target component data determined by the labeling from the first bio-signal data.

6. The method of claim 1, wherein the generating of the third bio-signal data comprises:
determining a weight of the noise component data based on a predetermined signal-to-noise ratio (SNR) value; and
generating the third bio-signal data by adding the noise component data multiplied by the weight to the second bio-signal data.

7. The method of claim 6, wherein the determining of the weight comprises:
determining the weight such that an SNR value of the third bio-signal data is equal to the predetermined SNR value.

8. A training method of a bio-signal denoising artificial intelligence (AI) model, the training method comprising:
receiving first bio-signal data measured in a non-noise-suppressed environment and second bio-signal data measured in a noise-suppressed environment;
performing component analysis of the first bio-signal data, and determining noise component data by separating target component data from the first bio-signal data;
generating third bio-signal data by combining the noise component data with the second bio-signal data;
determining the second bio-signal data as ground truth data of a training dataset of the denoising AI model and determining the third bio-signal data as noisy data of the training dataset to determine the training dataset;
generating fourth bio-signal data by executing the denoising AI model based on the noisy data and removing noise data included in the noisy data;
determining a loss value based on a difference between the ground truth data and the fourth bio-signal data or based on a difference between the noise component data and the noise data; and
training the denoising AI model based on the loss value.

9. The training method of claim 8, wherein the noise-suppressed environment is
an environment in which at least one of a bio-signal excluding a target bio-signal to be acquired by executing the denoising AI model among measured bio-signals, or external noise is suppressed, and
the target component data is
component data corresponding to the target bio-signal among component data of the first bio-signal data.

10. The training method of claim 8, wherein the component analysis corresponds to an independent component analysis (ICA).

11. The training method of claim 10, wherein the determining of the noise component data comprises:
separating the first bio-signal data by performing the ICA based on the number of predetermined independent component categories;
labeling the separated data based on the predetermined independent component categories; and
determining the noise component data by removing the target component data determined by the labeling from the first bio-signal data.

12. The training method of claim 8, wherein the generating of the third bio-signal data comprises:
determining a weight of the noise component data based on a predetermined signal-to-noise ratio (SNR) value; and
generating the third bio-signal data by adding the noise component data multiplied by the weight to the second bio-signal data.

13. The training method of claim 12, wherein the determining of the weight comprises:
determining the weight such that an SNR value of the third bio-signal data is equal to the predetermined SNR value.

14. A device for generating a training dataset of a bio-signal denoising artificial intelligence (AI) model, the device comprising:
one or more processors; and
memory including instructions executable by the one or more processors,
wherein the instructions, when executed by the one or more processors, cause the device to:
receive first bio-signal data measured in a non-noise-suppressed environment and second bio-signal data measured in a noise-suppressed environment,
perform component analysis of the first bio-signal data, and determine noise component data by separating target component data from the first bio-signal data,
generate third bio-signal data by combining the noise component data with the second bio-signal data, and
determine the second bio-signal data as ground truth data of the training dataset and determine the third bio-signal data as noisy data of the training dataset to determine the training dataset.

15. The device of claim 14, wherein the noise-suppressed environment is
an environment in which at least one of a bio-signal excluding a target bio-signal to be acquired by executing the denoising AI model among measured bio-signals, or external noise is suppressed, and
the target component data is
component data corresponding to the target bio-signal among component data of the first bio-signal data.

16. The device of claim 15, wherein the target bio-signal corresponds to one of an electroencephalogram (EEG), electrooculogram (EOG), electrocardiogram (ECG), and electromyogram (EMG).

17. The device of claim 14, wherein the component analysis corresponds to an independent component analysis (ICA).

18. The device of claim 17, wherein the instructions, when executed by the one or more processors, cause the device to, for the determining of the noise component data:
separate the first bio-signal data by performing the ICA based on the number of predetermined independent component categories,
label the separated data based on the predetermined independent component categories, and
determine the noise component data by removing the target component data determined by the labeling from the first bio-signal data.

19. The device of claim 14, wherein the instructions, when executed by the one or more processors, cause the device to, for the generating of the third bio-signal data:
determine a weight of the noise component data based on a predetermined signal-to-noise ratio (SNR) value, and
generate the third bio-signal data by adding the noise component data multiplied by the weight to the second bio-signal data.

20. The device of claim 19, wherein the instructions, when executed by the one or more processors, cause the device to, for the determining of the weight:
determine the weight such that an SNR value of the third bio-signal data is equal to the predetermined SNR value.
